(19) United States
(12) Patent Application Publication
(10) Pub. No.: US 2010/0184213 A1
(43) Pub. Date: Jul. 22, 2010

(54) APOCRINE CELL LINE

(76) Inventors: Jason Shaun Burry, Wirral (GB); Richard Livesey Evans, Wirral (GB); Mark Harker, Wirral (GB); George Terence Evelyn Kealey, Buckingham (GB); Donald Farquhar McDonald, London (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/532,438

(22) PCT Filed: Feb. 22, 2008
(86) PCT No.: PCT/EP08/52173

§ 371 Date: Feb. 1, 2010

(30) Foreign Application Priority Data
Mar. 26, 2007 (GB) 0705738.3

(51) Int. Cl. C12N 5/071

(52) U.S. Cl. 435/366

(57) ABSTRACT

An apocrine cell line is obtained by the steps of isolating an apocrine cell from primary tissue, culturing the isolated cell in a first culture medium, removing unattached cells from the first culture medium and transferring said unattached cells to a second culture medium comprising an effective concentration of a phorbol ester, and thereby establishing an apocrine cell line exhibiting long-term proliferation capability, which after many cultures is indicative of indefinite proliferation.

The present invention relates to an apocrine cell line and in particular to an apocrine cell line exhibiting long-term proliferation.

In addition to eccrine glands, apocrine glands secrete aqueous fluid containing various lipidic and aminoacid solutes which, when exposed on human skin to skin bacterial populations, are transformed to malodorous compounds, including steroids and short chain fatty acids. In other words, apocrine glands contribute to human sweating. Considerable research continues to be carried out to find means to counteract the generation of malodorous compounds on skin, but this is hampered by the absence of an apocrine cell line that closely mimics the functions of an apocrine gland in situ, i.e. in human skin. The development of the latter would be very valuable because it would offer one or more of the benefits mentioned herein. It would enable a greater number of in vitro, as opposed to in vivo, studies to be carried out. This would (i) assist in identifying the transport mechanisms within the apocrine gland that lead to sweating, (ii) facilitate the screening and identification of substances for reducing the formation of malodorous compounds, and (iii) help determine their effective concentration, i.e. the concentration required to act as a deodorant when applied topically to the body. The existence of a suitable cell line would also provide the opportunity to identify novel glandular functions other then sweating per se.

In common with attempts to sustain other secretory cells in long-term culture, primary cell lines obtained from apocrine cultures have not been passaged more than a few times before losing morphological, phenotypic and/or functional respects characteristic of an apocrine gland.

A paper by R Wicher et al in Andrologia 35, pp 342-350 entitled “Establishing of two in vitro models of epithelial cells from the apocrine secreting rat coagulating gland” relates to apocrine cells from rats, and does not disclose a human-derived apocrine cell line exhibiting long term proliferation. Furthermore, the cell markers used therein to demonstrate functionality are not necessarily relevant to the human apocrine model. Moreover, the authors seemingly did not seek to passage their cells after freezing and thawing, and thereby did not demonstrate their long term proliferation capability.

A paper by Z. Maras et al published in In Vitro Cell. Dev. Biol. Animal Vol 35, November-December (1999), pp 606-611 entitled “Cultivation of Epithelia from the Secretory Coil of the Ovine Apocrine Gland; Evidence of Secretory Cell Function and Ductal Morphogenesis in Vitro” relates to apocrine cells from sheep, and similarly does not disclose a human-derived apocrine cell line exhibiting long term proliferation.

A paper by Dieter C. Gruenert et al published in In Vitro Cell. Dev. Biol. Vol 26, April (1990), pp 411-416 entitled “Long Term Culture of Normal and Cystic Fibrosois

Epithelial Cells grown under Serum-free Conditions” relates to eccrine cells instead of apocrine cells and therefore does not disclose a human-derived apocrine cell line exhibiting long term proliferation.

### OBJECT OF THE PRESENT INVENTION

It is an object of the present invention to provide an apocrine cell line that mimics the sweating function of an apocrine gland, which can be maintained in culture through subcultures, and exhibits proliferation in the long term.

### BRIEF SUMMARY OF THE PRESENT INVENTION

According to the present invention there is provided a cultured apocrine cell line exhibiting long term proliferation.

Proliferation in the present context indicates that the cells retain the capability to divide when cultured in a suitable culture medium, but does not imply that the cell line is immortalised.

Long term, in the context of the present invention, indicates that cells retain their capability to proliferate after at least 30 sub-cultures (passages). In practice, when such long term proliferation has been achieved by a cell line, it can commonly exhibit proliferation through at least 100 or 1000 passages. Proliferation through such a large number of proliferations is indicative of indefinite proliferation, at least for practical purposes for cultured apocrine cell lines according to the present invention.

In particular, there is provided a human apocrine cell line ASG5 which has been deposited with the European Collection of Cell Cultures, (ECACC), Porton Down, Salisbury, SP4 0JG, England, under the depository number 07021301 and which exhibits at least long term proliferation.

### Detailed Description of Preferred Embodiments of the Invention and Morphology

This text describes the generation of a long term proliferative apocrine cell line, and subsequently, its characterisation and comparison with primary cultured apocrine cells via electrophysiological, molecular, and biochemical techniques. Particular interest is paid to steroid synthesis and the analysis of odour precursor compounds with express reference to the apocrine cell line ASG5, that has demonstrated at least long term proliferation.

### Isolation and Primary Culture of Apocrine Glands

An apocrine cell line that exhibited long term proliferation capability was obtained by the following method. Whole apocrine glands were isolated by shearing the axillary skin of women aged between 30 and 70, having previously obtained permission from the relevant ethical committee and informed consent of the subjects. Some apocrine sweat glands were fragmented by the shearing process, and cells could be cultured from those fragments. Microscopic observation and Neutral Red uptake experiments showed that the apocrine gland isolated by shearing consisted purely of coil separated from duct. On average between 10 and 100 apocrine coils were isolated from samples of axillary skin of about 50 mm×5 mm.

The apocrine coils were then incubated using a modified method, proposed by Lee et al in J. Cell Sci. 83:103-118, 1986, in a Williams E medium (herein for short WEM)-containing collagenase type II (at 2 mg ml⁻¹) for 30 minutes at 37° C. in 5% CO₂/air of 95% relative humidity. The coils were then washed in enzyme-free WEM supplemented with 10 ng ml⁻¹ epidermal growth factor (EGF), 10 μg ml⁻¹ insulin, 10 ng ml⁻¹ hydrocortisone, 10 μg ml⁻¹ holotransferrin, 1 mM L-glutamine, 100 U ml⁻¹ penicillin and 100 μg ml⁻¹ streptomycin.

Approximately 15 apocrine glands were plated out per 25 cm² flasks in 1 ml of Mammary Epithelial Growth Medium (MEGM), supplemented with 10 ng ml⁻¹ EGF, 10 μg ml⁻¹ insulin, 0.5 μg ml⁻¹ hydrocortisone, 30 μg ml⁻¹ bovine pituitary extract, 50 μg ml⁻¹ gentamicin and 50 ng ml⁻¹ amphotericin. After 24 hr incubation in a 95% air/5% CO₂ humidified incubator at 37° C., a further 3 ml MEGM was added to each flask and thereafter the medium was changed every 3 days.

### Generation and Maintenance of a Long Term Proliferating Apocrine Cell Line (ASG5)—Indicative of an Indefinitely Proliferating Cell Line

### Primary Sweat Gland Isolation and Culture

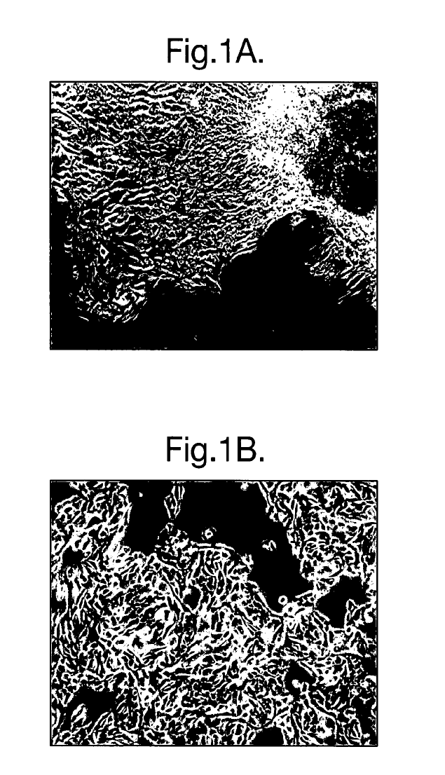

As indicated before, whole intact human apocrine sweat glands were isolated by shearing. Histological examination of apocrine glands revealed that shearing completely removed the short absorptive duct leaving only the secretory coil portion intact. Isolated glands were plated out in supplemented MEGM. Approximately 50% of glands plated out generated outgrowths, routinely observed after 2-3 days. Outgrowths continued to proliferate for up to 20 days and displayed typical ‘cobblestone’ epithelial cell morphology (as shown in FIG. 1, which show phase contrast micrographs of (A) cells growing from an apocrine coil nine days after explantation, and (B) proliferating apocrine cells). At early stages of culture, before outgrowths were observed, a small number of cells with an elongated fibroblastoid morphology were often observed around explanted glands. However, these cells did not proliferate in MEGM.

A proliferating apocrine cell line was derived from primary cultures of apocrine secretory coils. After 7 days culture in MEGM, the potent phorbol ester, TPA (phorbol-12-myristate-13 acetate; ex Calbiochem, Nottingham, UK) was included in the medium at a concentration of 250 nM. It is an important feature of the process of obtaining a cell-line exhibiting long term proliferation capability that the culture medium contains an effective concentration of an ester of phorbol, phorbol being alternatively called 1,1a,1b,4,4a,7a,7b,8,9,9a-decahydro-4-a,7b,9,9a-tetrahydroxy-3-(hydroxymethyl)-1,1,6,8-tetramethyl-5H-cyclopropa[3,4]benz[1,2-e]azulen-5-one. Commonly, the ester comprises at least aliphatic substituent particularly suitably at the 12 and/or 13 positions, such as a long chain alkyl (C₇ to C₂₄) and optionally also a short chain alkyl (C₂ to C₆).

Accordingly, the method herein enables the preparation of an apocrine cell line exhibiting indefinite proliferation.

TPA induced premature multilayering 3-4 days after addition. However, the upper differentiated layers were removed by repeated washing to leave a proliferative monolayer of cells still attached to the flask. Cells of the proliferative layer were passaged into 96 well plates to generate clonal cell lines. Cells were maintained, passaged and continued to grow in MEGM containing TPA for approximately 2 months. After this period cells were cultured in MEGM alone and monitored for continued proliferation. Initially, many subclones continued to grow, but by the end of the next 10 passages, the majority had begun to senesce. One culture, in particular, ASG5, continued to grow and has now been maintained in culture, proliferating over more than 40 passages with no signs of a reduction in growth rate. This is indicative of exhibiting a proliferation capability indefinitely. This culture continues to proliferate in the absence of TPA, can survive cryopreservation and is considered to be stable. By comparison, primary apocrine sweat gland cultures grow for a maximum of 1 month and 4 passages and do not survive cryopreservation. The cell culture passaged herein exhibiting long term proliferation is morphologically similar to primary apocrine cultures, for example peak agonist responses. Such a cell culture is accordingly capable of being used to demonstrate whether and/or the extent to which a substance exhibits deodorising properties in vitro, i.e. is capable of acting as a personal deodorant.

### Preparation of ASG5 Apocrine Cells for Electrophysiology

After 13-18 days, confluent ASG5 cultures were passaged onto Transwell collagen-coated permeable supports (0.33 cm² area, Costar, cat. no. 3495, High Wycombe, Bucks, UK). This involved incubation in 2% EDTA for 20 minutes, followed by trypsin (0.5%)-EDTA (0.2%) for approximately five minutes. 3×10⁵ cells were transferred onto each Transwell supplemented with WEM. The Transwell supports were suspended in 24-well plates in 0.6 ml of the same medium. For primary cultures, only first passage cells were used in experiments. The medium bathing cultures on Transwells was changed every two days.

### Electrophysiology

Transepithelial resistance (TER) across epithelia on Transwells was monitored using ‘chopstick’ electrodes (WPI, Alresford, Hants, UK) connected to an ohmmeter. The TER was estimated by subtracting the measurement for a moistened filter blank. At peak TER, the Transwell supports were mounted in an Ussing Chamber. Epithelia were bathed in a modified Krebs buffer, oxygenated and maintained at 37° C., pH 7.4, with 95% O₂/5% CO₂. The buffer consisted of (mM): NaCl, 117; KCl, 4.7; CaCl₂, 1.5; MgSO₄, 1.2; NaHCO₃, 25; and glucose, 11.1. Voltage and current electrodes (calomel and silver/silver chloride respectively, ABB Kent Taylor, Stonehouse, Glos, UK), connected to a voltage/current clamp were extended into the bath via bridges containing agar dissolved in 3 M potassium chloride.

Voltage electrodes were balanced in the absence of a Transwell and fluid resistance between the voltage electrodes was compensated for by the injection of an arbitrary DC current. After mounting a Transwell the epithelium was short-circuited, the current allowed to stabilise, typically for 15 minutes. Given the rarity of apocrine material, drugs were typically added in succession as disclosed by Brayden et al (1988) J. Physiol, 405: 657-675, Pedersen et al (1992) Exp. Physiol, 77; 863-871, and Shen et al, (1994) Am. J. Physiol, 266: L493-501 and changes in short circuit current (I^{sc}) recorded. The duration of experiments was typically 15-60 min. TER was estimated using Ohm's Law, routinely by clamping the voltage at ±10 mV and measuring the current required to achieve this. Unless otherwise stated all drugs listed in the legend were added to the basolateral side, except for amiloride that was added to the apical side. Amiloride is an inhibitor of the epithelial Na⁺ channel (ENaC) and the Na⁺/H⁺ exchanger.

### Modifications of Krebs Buffer

Chloride substitution experiments were carried out in Krebs buffer in which sodium gluconate was substituted for NaCl, potassium gluconate for KCl and CaSO₄ for CaCl₂. Glucose-free experiments were carried out with 11.1 mM mannitol as a replacement to maintain osmolality. Experiments in which barium was used as an inhibitor of potassium channels were carried out in the absence of sulphate ions, such that MgCl₂ replaced MgSO₄.

### Basal Transepithelial Properties

Proliferating apocrine cultures were passaged onto permeable supports and transferred to an Ussing chamber at peak TER. The mean TER for such proliferating cultures was 395±40 Ωcm² (P<0.001). Resting short circuit current (I^{sc}) was 4.5±0.8 μA cm⁻² (P<0.001) for proliferating apocrine cultures. Resting transepithelial potential difference (TEPD) was 1.0 mV for proliferating apocrine cultures, apically negative.

### Effects of Ion Transport Inhibitors on Resting I^{sc} Summarised in Table 1 Below.

Resting I^{sc} in proliferating cultures was sensitive to 10 μM amiloride confirming that sodium reabsorption is a significant component of ion transport in cultured sweat gland epithelia. Apically applied amiloride (10 μM) reduced resting I^{sc} by 4.5±1.0 μA cm⁻² in proliferating apocrine cultures (n=20, P<0.001). Dose response curves reveal that the IC₅₀ for amiloride is 2-4 μM and that 10 μM amiloride was a supramaximal dose.

The effects of furosemide, an inhibitor of NKCC1, the epithelial Na⁺—K⁺—Cl⁻ cotransporter (and hence transepithelial chloride transport) was also examined in apocrine cultures. Basolateral addition of 1 mM furosemide reduced resting I^{sc} in transformed cultures by 0.8±0.30.2 μA (n=20, P<0.001).

TABLE 1
The effects of amiloride and furosemide on the
short circuit current in cultured apocrine (primary
and proliferating) and eccrine sweat gland cells.

| | Primary | Proliferating | Primary |
|---|---|---|---|
| | Apocrine | Apocrine | Eccrine |
| Resting I^{sc} | 8.4 ± 0.9 | 4.5 ± 0.8 | 8.6 ± 1.0 |
| (μA cm⁻²) | (n = 57) | (n = 40) | (n = 22) |
| ΔI^{sc} (μA cm⁻²) | | | |
| Amiloride | −6.0 ± 0.9** | −4.2 ± 1.0 | −7.1 ± 0.7 |
| (10 μM) | (n = 49) | (n = 20) | (n = 22) |
| Furosemide | −0.5 ± 0.2 | −0.8 ± 0.3 | nd |
| (1 mM) | (n = 13) | (n = 20) | |

### The Role of Sodium Reabsorption in Agonist Responses

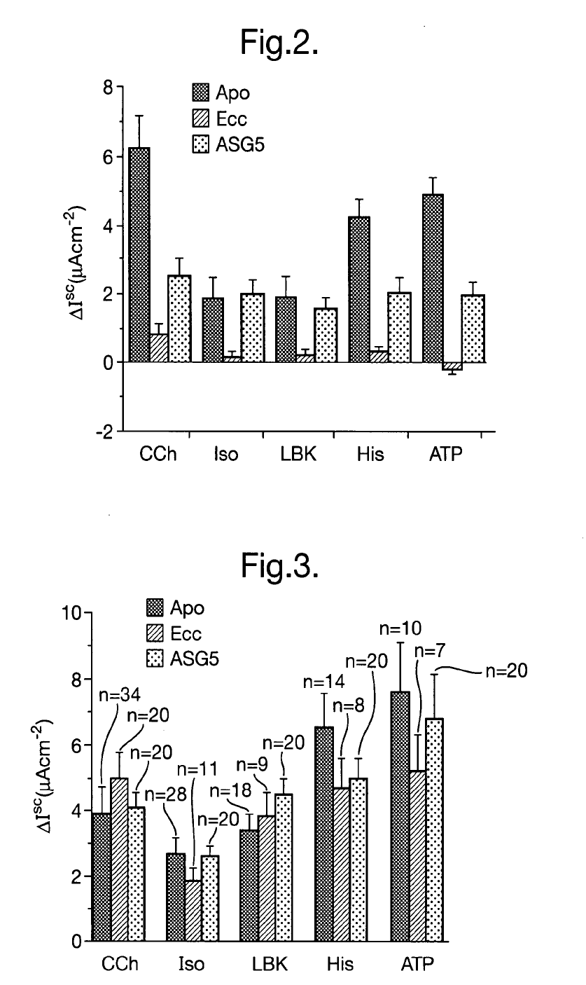

Primary apocrine and proliferating apocrine cultures were pre-treated with 10 μM amiloride to determine the role of sodium reabsorption in agonist responses. Appended FIG. 2 shows Peak increases in I^{sc} in primary apocrine, eccrine and proliferating apocrine cultures in the presence of amiloride. The agonists were added to the basolateral side of cultures on Transwells. Each bar in the Figure represents a minimum of n=10. Peak increases in short circuit current in the presence of apical amiloride (10 μM) are shown. The agonists were added basolaterally: Carbachol (CCh; 20 μM), Isoprenaline (Iso; 10 μM), L-bradykinin (LBK; 170 nM), Histamine (His; 200 μM) and Adenosine triphosphate (ATP; 100 μM). Significance was tested using unpaired Student's t test and the Wilcoxon tests.

FIG. 2 demonstrates that amiloride pre-treatment significantly reduced responses to CCh, His and ATP in a long term proliferating apocrine cell line, ASG5, suggesting that sodium reabsorption is a component of these responses.

FIG. 3 shows peak increases in I^{sc} in primary apocrine, eccrine and proliferating apocrine cultures. Agonists were added to the basolateral side of cultures on Transwells. Peak increases in short circuit current are shown. The agonists were added basolaterally: CCh (20 μM), Iso (10 μM), LBK (170 nM), His (200 μM) and ATP (100 μM).

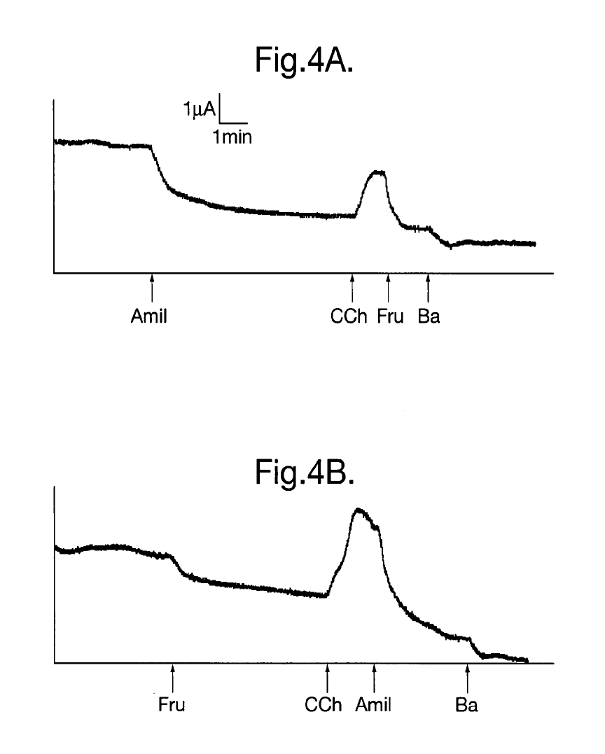

FIGS. 4A and 4B are representative I′ records (traces) on the effects of ion channel inhibitors on the response to carbachol in ASG5 cells. FIG. 4A shows an apical pre-treatment with amiloride (10 μM) and the effects of addition of 1 mM furosemide (Fru) and 50 μM barium chloride (Ba; a maxi-K channel blocker), on the CCh (20 μM) response. FIG. 4B shows an apical pre-treatment with 1 mM furosemide and the effects of amiloride and barium chloride on CCh response.

The representative trace of proliferating apocrine cultures in FIG. 4A also demonstrated that compared to the observed values for agonist stimulation in FIG. 3, carbachol stimulation is reduced after amiloride pre-treatment.

In four cases, whole apocrine secretory coils were explanted directly onto permeable supports to determine if the primary culture and passage of apocrine cultures affected their electrophysiological properties. In the cultures examined, basal and agonist stimulated electrophysiological properties were not significantly different to primary cultures, whether in the presence or absence of amiloride (data not shown).

### The Role of Transepithelial Chloride Transport in Transient Agonist Responses

To further characterise the ionic basis of agonist induced responses in primary and proliferating apocrine cultures, the role of transepithelial chloride transport was examined. Under chloride-free conditions, resting I′ for primary and proliferating apocrine cultures was 6.0±0.8 μA cm⁻² and 3.1±0.6 μA cm⁻² respectively. Resting TEPD was −0.9 mV for primary cultures and −1.8 mV for proliferating cultures. Addition of 10 μM amiloride reduced basal I^{sc} by 3.6 μA cm⁻² in primary cultures and by 2.5 μA cm⁻² in proliferating cultures.

Responses to carbachol, histamine and ATP were significantly reduced in both primary and proliferating apocrine cultures when compared to identical experiments in the presence of chloride (Table 2) suggesting that chloride transport does play a significant role in mediating agonist responses. Furthermore, FIG. 4B shows a representative trace from proliferating cultures demonstrating that pre-treatment with furosemide reduces the magnitude of response to carbachol compared to the values in FIG. 3. Addition of furosemide after agonist stimulation induced a rapid reduction in responses in both primary and transformed apocrine cultures, demonstrating the role of NKCC1 in regulating chloride uptake. (Table 3).

TABLE 2
Peak agonist responses of apocrine cultures in
chloride-free buffer.

| Agonist | | | | |
|---|---|---|---|---|
| I^{sc} responses μA cm⁻² | Chloride-free | n | Control | n |
| A | | | | |
| Carbachol (20 μM) | 0.7 ± 0.6* | 5 | 3.9 ± 0.7 | 34 |
| Histamine (200 μM) | 1.7 ± 0.5** | 5 | 6.4 ± 1.3 | 14 |
| ATP (100 μM) | 2.9 ± 0.9* | 9 | 7.4 ± 1.8 | 10 |
| B | | | | |
| Carbachol (20 μM) | 3.0 ± 0.8* | 6 | 4.0 ± 0.7 | 20 |
| Histamine (200 μM) | 3.3 ± 0.8** | 6 | 4.8 ± 1.3 | 20 |
| ATP (100 μM) | 4.0 ± 0.5* | 6 | 6.3 ± 1.8 | 20 |

A: Primary apocrine cultures
B: proliferating apocrine culture (ASG5).

TABLE 3
Furosemide inhibition of agonist responses in primary
and long term proliferating (AGS5) apocrine cultures.

Inhibition by furosemide (90 μM)
Δ I^{sc}, μA cm⁻²

| Agonist | Primary | Proliferating |
|---|---|---|
| Carbachol (≧20 μM) | −2.0 ± 0.5 | −1.1 ± 0.4 |
| | (n = 4) | (n = 10) |
| Isoprenaline (10 μM) | 0.8 ± 0.2 | −1.2 ± 0.3 |
| | (n = 3) | (n = 10) |
| Histamine (200 μM) | −1.3 ± 0.2 | −1.0 ± 0.4 |
| | (n = 6) | (n = 10) |
| ATP (100 μM) | −2.6 ± 0.5 | −1.2 ± 0.3 |
| | (n = 8) | (n = 10) |

Agonist responses were significantly reduced with amiloride pre-treatment when compared to amiloride pre-treatment in chloride containing buffer, but they were not entirely abolished, particularly in the proliferating cultures, suggesting that other ion transport pathways may also play a role in these responses (Table 4).

TABLE 4
Peak agonist responses of apocrine cultures in
chloride-free buffer after amiloride pre-treatment.

| Agonist | | | | |
|---|---|---|---|---|
| I^{sc} responses μA cm⁻² | Chloride-free | n | Control | N |
| A | | | | |
| Carbachol (200 μM) | 1.7 ± 1.2** | 9 | 6.3 ± 1.4 | 11 |
| Isoprenaline (≧1 μM) | 0.7 ± 0.3* | 15 | 1.8 ± 0.4 | 20 |
| LBK (170 nM) | 0.6 ± 0.4* | 11 | 2.0 ± 0.5 | 18 |
| Histamine (200 μM) | 2.4 ± 1.4 | 8 | 4.2 ± 0.5 | 19 |
| ATP (100 μM) | 4.3 ± 2.4 | 6 | 4.9 ± 0.7 | 24 |
| B | | | | |
| Carbachol (200 μM) | 1.3 ± 0.3 | 6 | 2.4 ± 0.6 | 20 |
| Isoprenaline (≧1 μM) | 0.8 ± 0.2 | 6 | 1.8 ± 0.4 | 20 |
| LBK (170 nM) | 0.9 ± 0.3 | 6 | 1.6 ± 0.3 | 20 |
| Histamine (200 μM | 1.0 ± 0.2 | 6 | 1.9 ± 0.4 | 20 |
| ATP (100 μM) | 0.8 ± 0.2 | 6 | 1.8 ± 0.3 | 20 |

A: Primary apocrine cultures
B: proliferating apocrine cultures.

Taken together these results indicate that the proliferating cell line continued to exhibit the same characteristics as the primary cultures.

### Steroidogenesis in a Long Term Proliferating Apocrine Gland Cell Line

Human skin and its associated appendages are capable of a variety of metabolic functions, including those involved in the metabolism and synthesis of hormones. Androgens (sex steroid hormones), are synthesized from the parental precursor cholesterol in a biosynthetic pathway requiring numerous enzymatic conversions. As apocrine glands become functional during puberty, it is likely that androgens play some role in modulating gland activity. This is supported further by the fact that the androgen receptor has been detected in the secretory cells of apocrine glands residing in axillary skin (Beier et al, (2005) Histochem. Cell Biol., 123: 61-65). Since the local or intracrine formation of steroids potentially plays an important role in mediating cell function (Labrie, F. et al, (1991), Mol. Cell. Endocrinol., 78: 113-118), the expression of transcripts responsible was investigated for the formation of active androgens and estrogens in the indefinitely proliferating apocrine cell line herein, as well as their associated receptors.

### Materials & Methods in the Investigation

### Chemicals

Taq DNA polymerase and molecular marker III were purchased from Roche, Mannheim Germany. Taq DNA polymerase was used according to the suppliers' recommendations. All other reagents were obtained from Sigma-Aldrich, Gillingham, UK.

### Strains, Plasmids, Media and Culture Conditions

E. coli strain TOP10 (Invitrogen, Carlsbad, Calif., USA) was used as the host strain in all cloning procedures. Pre-digested vector pCR®4-TOPO® containing poly-T overhangs was obtained from Invitrogen and used according to the suppliers instructions. Bacteria were cultivated in LB medium (10 g/l tryptone, 5 g/l yeast extract, 5 g/l NaCl) supplemented with the appropriate selection pressure (kanamycin 50 μg/ml) on a rotary shaker (250 rpm) at 37° C.

### Cell Material

Long term proliferating apocrine gland cells indicative of indefinite proliferation, were grown as described hereinbefore. For cloning purposes cells were harvested after 10 days growth at 37° C.

### Oligonucleotide Synthesis and Sequencing

All oligonucleotides used were synthesized by Sigma-Genosys Ltd, Pampisford, UK. Sequencing was performed using an ABI Prism 3100 Genetic Analyser using the universal M13 forward and reverse primers.

### Isolation of Plasmid DNA

The Qiagen (Crawley, UK) mini-prep kit was used to obtain plasmid DNA for cloning and sequencing.

### Cloning

Fragments of transcripts involved in steroid androgen metabolism were cloned from cDNA derived from indefinitely proliferating apocrine gland cells. RNA was isolated from the cells using the RNAqueous™-4 PCR kit from Ambion, Huntingdon, UK. First strand cDNA synthesis was performed using the Ambion RETROscript™ kit using oligo(dt). The PCR reaction programme used for amplification was: 1 cycle (120 s @ 95° C.), 30 cycles (30 s @ 95° C., 30 s @ 60° C., 45 s or 60 s @ 72° C.), 1 cycle (7 min @ 72° C.) with Taq DNA polymerase). The primers used to clone fragments from each transcript are outlined in Table 5.

TABLE 5
Primers used to clone gene fragments involved
in androgen metabolism.

| | | Ex- |
|---|---|---|
| | | pected |
| | | size |
| Primer sequence | Gene | (bp) |
| Forward 5′- | Steroid | 392 |
| CTTCACCCCCAACTTCAACCCCG-3′ | sulphatase | |
| Reverse 5′- | | |
| CCA CAT GCG TCT GTC TGG TCC | | |
| CC-3′ | | |
| Forward 5′- | 3β-hydroxy-Δ- | 813 |
| GCCAGTCTTCATCTACACCAGTAG C-3′ | 5-steroid | |
| Reverse 5′- | dehydrogenase | |
| CTCTGTCATCCTTAAATCACTGAGTC-3′ | 1 | |
| Forward 5′- | Hydroxysteroid | 436 |
| CCTCATCCATTGTAACATCACCTCC-3′ | 17β- | |
| Reverse 5′- | dehydrogenase | |
| CTCACCGCCTGGCTACCTGACC-3′ | 3 | |
| Forward 5′- | Hydroxysteroid | 990 |
| GACAAGTGACAGGGAATGGATTCC-3′ | 17β- | |
| Reverse 5′- | dehydrogenase | |
| CAGGGCTTCTGGTAGACATCAGG-3′ | 5 | |
| Forward 5′- | Hydroxysteroid | 722 |
| GAAGTGTGAGTGCGCGAAGATGCG-3′ | 17β- | |
| Reverse 5′- | dehydrogenase | |
| GTGCTGGAATTATAGGCATGAGCCAC-3′ | 7 | |
| Forward 5′- | 5α Reductase | 773 |
| CCAGCCCTGGCGATGGCAACG-3′ | | |
| Reverse 5′- | | |
| GAAATTCTGACCTGTTACACAGTAGG-3′ | | |
| Forward 5′- | Aromatase | 447 |
| GCCTGTCGTGGACTTGGTCATGCG-3′ | | |
| Reverse 5′- | | |
| GAGTAGGTACTGACCAGCCTTCTC-3′ | | |
| Forward 5′- | Estrogen | 564 |
| CTTGGAGAGCTGTTGGATGGAGGTG-3′ | receptor β | |
| Reverse 5′- | | |
| CAGGGCCAGGCGTCACTGAGA | | |
| C-3′ | | |
| Forward 5′- | Androgen | 564 |
| CTGGATGGGGCTCATGGTGTTTG-3′ | receptor | |
| Reverse 5′- | | |
| GTTTCCAATGCTTCACTGGGTGTGG-3′ | | |

All the gene fragments that primer pairs were designed for, as outlined in table 5, could be detected. Appended FIG. 5 displays a 1.2% agarose gel of all the PCR products produced.

FIG. 5 shows the expression of gene fragments:

1. Steroid sulphatase; 2. 3βhydroxy-Δ-5-steroid dehydrogenase 1; 3. Hydroxysteroid 17β-dehydrogenase 3; 4. Hydroxysteroid 17β-dehydrogenase 5; 5. Hydroxysteroid 17β-dehydrogenase 7; 6. 5α Reductase I; 7. CYP19A1 Aromatase; 8. Androgen receptor; 9. Estrogen receptor β; M. DNA Marker III.

These PCR products were then cloned into the TOPO vector ready for sequencing. Sequencing was performed on each PCR product to confirm the identity of each cloned fragment. The results of the gene sequencing are given below.

Steroid sulphatase fragment sequence
TCCCTTCTTCACCCCCAACTTCAACCCCGTGGGTTCCAACGGATGCTTTG
CCACACACGTGTGCTTCTGTTTCGGGAGTTATGTCACCCATCACGACCCA
CCTTTACTCTTTGATATTTCCAAAGATCCCAGAGAGAGAAACCCACTAAC
TCCAGCATCCGAGCCCCGGTTTTATGAAATCCTCAAAGTCATGCAGGAAG
CTGCGGACAGACACACCCAGACCCTGCCAGAGGTGCCCGATCAGTTTTCA
TGGAACAACTTTCTTTGGAAGCCCTGGCTTCAGCTGTGCTGTCCTTCCAC
CGGCCTGTCTTGCCAGTGTGATAGAGAAAAACAGGATAAGAGACTGAGCC
GCTAGCAGCGCCTGGGGACCAGACAGACGCATGTGGAAGGGC
3β-hydroxy-Δ-5-steroid dehydrogenase 1 fragment
sequence
GCCCTTCTCTGTCATCCTTAAATCACTGAGTCTTGGACTTCACGGTCTCC
TTGTGCCAGTCCACAAGGGAAACCCACTCCACGGTTCTAACAGACTCAGG
TACATTCTTCTTGGCTTCCTCCCAGCTGTAAAGTGGCTTATATGCCAGAT
CTTGCTGAGCCTTCTTGTAAGAGAAGGTGAACACGCTATTTGACAATGTC
ACTCTGTGGTGGTTGAAGGGTGTCGATAGGTGTAAATTGGCCTCAGCAGG
AAGCTCACTATTTCCAGCAGGAAGCCAATCCAGTACATCAGAGATAAAGG
AAGGCTCCATCTAGAATCAAGGCAGAGGCCGAACTCTTTGCTCAGGATGT
AATTAAGGTTATCATAGCTTTGGTGAGGCATGTCATCTGAGATGTAGTAG
AACTGTCCTCGGACACTTGGGGCCTTCTTGGGCTCCCGCAGGGCCTCAAG
GCCAGAATGTGGGCCCAGGCCATGTTTCCAATATAGACTGGGTTGACTCT
GGAGAACTTGCTGAAACTTGACAGGATCCCATGGTTGTTTAGGGCCTCAT
TTATACCGGCAGAAAGGATTGGGCTTCCTTCCCCATAGATAAACATTGGT
CTTAAGGCACAAGTGTACAAGGTGCCACCGTTTTTCAGAGTCCACCCATT
AGCCGCCAGCACAGCCTTCTCAGCAAGCTTTTTGCTGTGTGGGTATGGAG
CGTACCATGTGTTTTCCAGAGGCTCTTCTTCGTGACTGTTCTGGATGATT
TCCTTGTAGGAATTGGGCCCGGCTACCTCTGGGCTACTGGTGTAGATGAA
GACTGGCAAGGGC
Hydroxysteroid 17β-dehydrogenase 3 fragment
sequence
GCCCTTCTCACCCNCTGGCTACCTGACCTTGGTGTTGAGCTTCAGGTATG
CCACATAGTGTGTCAGGAGCAGCCTTTGGAAGGCACCGCTGTAGAAGGCC
CAGGCCGGGATCAGGCTCAGAAAGCCCGCCAAGATTTCATGGGCAAGGCA
GCCGCAGGTTTCACCTCCAATTGTGACATAATTCAATGACTCTTTGACAA
ACTCATCAGCAGTCTTGGTTATCACATTTGTATTTAGATACTTTGTCATT
GCAGTCGGGACAGCATATGGGGTCAGCACCTGGATGATGACTTCTTTTGC
TTTATATTCCTCTTGCAGGGCCTTGGAAAATGCGCACACAAACGCCTTGG
AAGCTGAGTACATGGAGTAGAGAGGCCAAGGAAACAGGGCTATCCCAGAA
GAAATGTTCAGGATGAGACCTTTCTGCCTTGATTCCATATGTTTCAAAAT
TAGCTGTGTCATCTTGACTACGGAGGTGATGTTACAATGGATGAGGAAGG
GC
Hydroxysteroid 17β-dehydrogenase 5 fragment
sequence
GACAAGTGACAGGGAATGGATTCCAAACACCAGTGTGTAAAGCTAAAGGA
TGGCCACTTCATGCCTGTATTGGGATTTGGCACCTATGCACCTCCAGAGG
TTCCAAGAAGTAAAGCTTTGGAGGTCACAAAATTAGCAATAGAAGCTGGG
TTCCGCCATATAGATTCTGCTCATTTATACAATAATGAGGAGCAGGTTGG
ACTGGCCATCCGAAGCAAGATTGCAGATGGCAGTGTGAAGGGAGAAGACA
TATTCTACACTTCAAAGCTTTGGTCCACTTTTCATCGACCAGAGTTGGTC
CGACCAGCCTTGGAAAACTCACTGAAAAAAGCTCAATTGGACTATGTTGA
CCTCTATCTTATTCATTCTCCAATGTCTCTAAAGCCAGGTGAGGAACTTT
CACCAACAGATGAAAATGGAAAAGTAATATTTGACATAGTGGATCTCTGT
ACCACCTGGGAGGCCATGGAGAAGTGTAAGGATGCAGGATTGGCCAAGTC
CATTGGGGTGTCAAACTTCAACCGCAGGCAGCTGGAGATGATCCTCAACA
AGCCAGGACTCAAGTACAAGCCTGTCTGCAACCAGGTAGAATGTCATCCG
TATTTCAACCGGAGTAAATTGCTAGATTTCTGCAAGTCGAAAGATATTGT
TCTGGTTGCCTATAGTGCTCTGGGATCTCAACGAGACAAACGATGGGTGG
ACCCGAACTCCCCGGTGCTCTTGGAGGACCCAGTCCTTTGTGCCTTGGCA
AAAAAGCACAAGCGAACCCCAGCCCTGATTGCCCTGCGCTACCAGCTGCA
GCGTGGGGTTGTGGTCCTGGCCAAGAGCTACAATGAGCAGCGCATCAGAC
AGAACGTGCAGGTTTTTGAGTTCCAGTTGACTGCAGAGGACATGAAAGCC
ATAGATGGCCTAGACAGAAATCTCCACTATTTTAACAGTGATAGTTTTGC
TAGCCACCCTAATTATCCATATTCAGATGAATATTAACATGGAGAGCTTT
GCCTGATGTCTACCAGAAGCCCTG
Hydroxysteroid 17β-dehydrogenase 7 fragment
sequence
GCCCTTGAAGTGTTAGTGCGCGAAGATGCGAAAGGTGGTTTTGATCACCG
GGGCTAGCAGTGGCATTGGCCTGGCCCTCTGCAAGCGGCTGCTGGCGGAA
GATGATGAGCTTCATCTGTGTTTGGCGTGCAGGAACATGAGCAAGGCAGA
AGCTGTCTGTGCTGCTCTGCTGGCCTCTCACCCCACTGCTGAGGTCACCA
TTGTCCAGGTGGATGTCAGCAACCTGCAGTCGGTCTTCCGGGCCTCCAAG
GAACTTAAGCAAAGGTTTCAGAGATTAGACTGTATATATCTAAATGCTGG
GATCATGCCTAATCCACAACTAAATATCAAAGCACTTTTCTTTGGCCTCT
TTTCAAGAAAAGTGATTCATATGTTCTCCACAGCTGAAGGCCTGCTGACC
CAGGGTGATAAGATCACTGCTGATGGACTTCAGGAGGTGTTTGAGACCAA
TGTCTTTGGCCATTTTATCCTGATTCGGGAACTGGAGCCTCTCCTCTGTC
ACAGTGACAATCCATCTCAGCTCATCTGGACATCATCTCGCAGTGCAAGG
AAATCTAATTTCAGCCTCGAGGACTTCCAGCACAGCAAAGGCAAGGAACC
CTACAGCTCTTCCAAATATGCCACTGACCTTTTGAGTGTGGCTTTGAACA
GGAACTTCAACCAGCAGGGTCTCTATTCCAATGTGGCCTGTCCAGGTACA
GCATTGACCAATTTGACATATGGAATTCTGCCTCCGTTTATATGGACGCT
GTTGATGCCGGCAATATTGCTACTTCGCTTTTTTGCAAATGCATTCACTT
TGACACCATATAATGGAACAGAAGCTCTGGTATGGCTTTTCCACCAAAAG
CCTGAATCTCTCAATCCTCTGATCAAATATCTGAGTGCCACCACTGGCTT
TGGAAGAAATTACATTATGACCCAGAAGATGGACCTAGATGAAGACACTG
CTGAAAAATTTTATCAAAAGTTACTGGAACTGGAAAAGCACATTAGGGTC
ACTATTCAAAAAACAGATAATCAGGCCAGGCTCAGTGGCTCATGCCTATA
ATTCCAGCAC
5α Reductase I fragment sequence
GCCCTTCCAGCCCTGGCGATGGCAACGGCGACGGGGGTGGCGGAGGAGCG
CCTGCTGGCCGCGCTCGCCTACCTGCAGTGCGCCGTGGGCTGCGCGGTCT
TCGCGCGCAATCGTCAGACGAACTCAGTGTACGGCCGCCCCGCGTCTCCG
CAGCGCGCCCAACTGCATCCTCCTGGCCATGTTCCTCGTCCACTACGGGC
ATCGGTGCTTAATTTACCCATTTCTGATGCGAGGAGGAAAGCCTATGCCA
CTGTTGGCGTGTACAATGGCGATTATGTTCTGTACCTGTAACGGCTATTT
GCAAAGCAGATACTTGAGCCATTGTGCAGTGTATGCTGATGACTGGGTAA
CAGATCCCCGTTTTCTAATAGGTTTTGGCTTGTGGTTAACGGGCATGTTG
ATAAACATCCATTCAGATCATATCCTAAGGAATCTCAGAAAACCAGGAGA
TACTGGATACAAAATACCAAGGGGAGGCTTATTTGAATACGTAACTGCAG
CCAACTATTTTGGAGAAATCATGGAGTGGTGTGGCTATGCCCTGGCCAGC
TGGTCTGTCCAAGGCGCGGCTTTTGCTTTCTTCACGTTTTGTTTTTTATC
TGGTAGAGCAAAAGAGCATCATGAGTGGTACCTCCGGAAATTTGAAGAGT
ATCCAAAGTTCAGAAAAATTATAATTCCATTTTTGTTTTAAGTGCGTTTT
TCATGAAATTATCTTCAACTTGAAGCTTTCCAATGGCGCTTCTCTATGGA
CTTTGTAAATAAGTTATATCTTTGTAATTTTCCTGCTACTTTATCATTTT
CAAGATGTCCTCTAGGAATTTTTTTTCTAGTAATTTTGCAATCTACCTAA
TAAGTACCTAAATACGCTGAAATGGAGGTTGAATATCCTACTGTGTAACA
GGTCAGAATTNCANGGGC
CYP19A1 Aromatase fragment sequence
GCCCTTGAGTAGNTACTGACCAGCCTTCTCTAGTGTTCCAGACACCTGTC
TGAGTTTCTTGGGGTAAAGATCATTTCCAGCATGTTTTTAGTCTCATCTG
GGTGCAAGGACAAGTCGTGTATCTTCTGTATGCTCTCAACACACTGTCCT
TGCAATGTCTTCACGTGGAATCGTCTCAGAAGTGTAACGAGGATGGCTTT
CATCATCACCATGGCGATGTACTTTCCTGCACAGCCACGGGGCCCAAAGC
CAAATGGCTGAAAGTACCTATAAGGAACATTCTTTGCAAAATTTTCAAGA
GTAAATTCATTGGGTTTGGGGAAAAACTCGAGTCTGTGCATCCTTCCAAT
ATTCAGGATAATGTTTGTCCCCTTTTTCACTGGGTAGCCATCGATTACAT
CATCTTCTAAGGCTTTGCGCATGACCAAGTCCACGACAGGCAAGGGC
Androgen receptor fragment sequence
GCCCTTGTTTCCNTGCTTCACTGGGTGTGGAAATAGATGGGCTTGACTTT
CCCAGAAAGGATCTTGGGCACTTGCACAGAGATGATCTCTGCCATCATTT
CCGGAAAGTCCACGCTCACCATGTGTGACTTGATTAGCAGGTCAAAAGTG
AACTGATGCAGCTCTCTCGCAATAGGCTGCACGGAGTCCAGGAGCTTGGT
GAGCTGGTAGAAGCGTCTTGAGCAGGATGTGGGATTTTTTCTTTTGCATG
CAATGATACGATCGAGTTCCTTGATGTAGTTCATTCGAAGTTCATCAAAG
AATTTTTGATTTTTCAGCCCATCCACTGGAATAATGCTGAAGAGTAGCAG
TGCTTTCATGCACAGGAATTCCTGGGGGGTGATTTGGAGCCATCCAAACT
CTTGAGAGAGGTGCCTCATTCGGACACACTGGCTGTACATCCGGGACTTG
TGCATGCGGTACTCATTGAAAACCAGATCAGGGGCGAAGTAGAGCATCCT
GGAGTTGACATTGGTGAAGGATCGCCAGCCCATGGCAAACACCATGAGCC
CCATCCAGAAGGGC
Estrogen receptor β fragment sequence
GCCCTTCAGGGNCNNGGCGTCACTGAGACTGTGGGTTCTGGGAGCCCTCT
TTGCTTTTACTGTCCTCTGCCGGGCTGCACTCGGACCCCGTGATGGAGGA
CTTGCACCCGCGAAGCACGTGGGCATTCAGCATCTCCAGCAGCAGGTCAT
ACACTGGGACCACATTTTTGCACTTCATGTTGAGCAGATGTTCCATGCCC
TTGTTACTCGCATGCCTGACGTGGGACAGGAGCATCAGGAGGTTAGCCAG
GCGCATGGATTGCTGCTGGGAGGAGATGCCGCTCTTGGCAATCACCCAAA
CCAAAGCATCGGTCACGGCGTTCAGCAAGTGAGCCAGCTTCCGGCTGCTG
TCAGCATCCTGGGTCGCTGTGACCAGAGGGTACATACTGGAATTGAGCAG
GATCATGGCCTTGACACAGAGATATTCTTTGTGTTGGAGTTTTAACTCTC
GAAACCTTGAAGTAGTTGCCAGGAGCATGTCAAAGATTTCCAGAATTCCT
TCTACGCATTTCCCCTCATCCGTCTGTAATCCCAACAATTTGGGAGGCTG
AGGCTGGGGGATCACTTGAAGTCAGGACCTCGAGACCAGCTTGGCCAACA
TGGTAAAACCCCGTCTCCACTGTCCAGAACAAGATCTGGAGCAAAGATGA
GCCTGCCGGGGTGGTCAATTGAGCGCCACATCAGCCCCATCATTAACACC
TCCATCCAACAGCTCTCCAAGAAGGGC

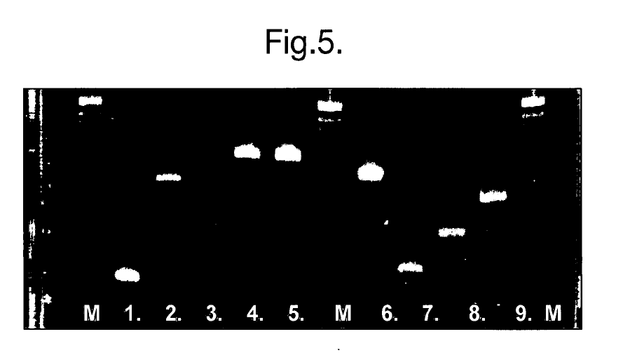

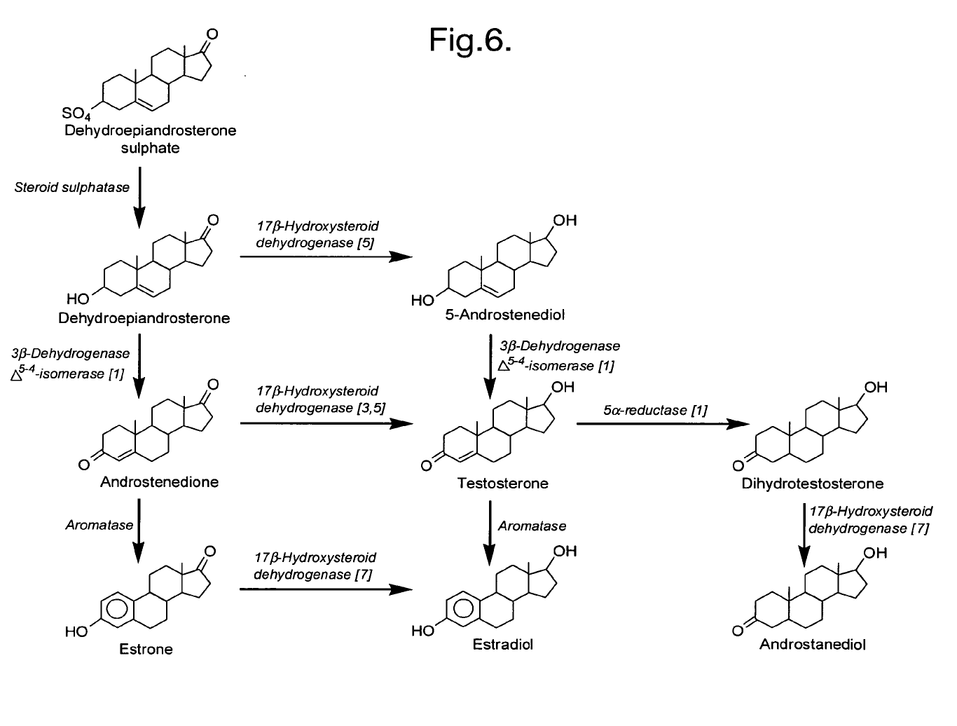

These results clearly demonstrate that the indefinitely proliferating apocrine gland cell line is expressing numerous genes required for androgen and estrogen synthesis. Appended FIG. 6 outlines the steroid synthesis pathway that can be elucidated given the genes that have shown to be expressed in the indefinitely proliferating apocrine gland cell line. In addition the cells also express the necessary receptors required to mediate the intracellular response to the production of these androgens and estrogens i.e. the androgen and estrogen β receptor. Interestingly, no transcript for the expression of the estrogen α receptor could be detected (data not shown). This is in agreement with a previous study that demonstrated that the estrogen β receptor could be detected in apocrine gland secretory cells in axillary skin sections but not the estrogen a receptor, using immunohistochemical staining for both receptor types (Beier, K. et al (2005) Histochem. Cell Biol., 123: 61-65). The same study also demonstrated the presence of the androgen receptor in apocrine gland secretory cells.

The data in appended FIG. 6 demonstrates that the indefinitely proliferating apocrine cell line retains many of the steroidogenic features of secretory apocrine gland cells observed in-vivo and primary culture. This cell line provides an excellent tool for studying steroid metabolism in apocrine glands and the role of steroids in regulating gland activity. Additionally, the cell line will serve as an invaluable tool for evaluating the efficacy of compounds in regulating gland activity via interference of the steroidogenic pathways in these cells.

### Cell Markers of Apocrine Secretory Activity in the Indefinitely Proliferating Apocrine Gland Cell Line.

The major function of the apocrine sweat gland is to produce a lipid rich secretion which is delivered to the skin surface via the canal of the hair follicle in direct response to emotional stimuli. The secretion is non-odorous, but undergoes bacterial decomposition which results in the generation of axillary malodours. The secretory function of the apocrine glands is associated with a number of proteins involved in these key processes.

The ABCC11 protein belongs to the family of ATP-binding cassette transporters involved in the efflux of purine and pyrimidine nucleotide analogs such as cAMP and cGMP (Guo et al., (2003) J. Biol. Chem., 278: 29509-29514). This protein has been implicated in the secretion of earwax in the ceruminous apocrine glands of humans (Yoshiura et al., (2006) Nat. Gen., 38: 324-330). Human earwax normally consists of dry and wet types. Dry earwax is frequent in East Asians, whereas wet earwax is common in other populations. A SNP, 538GA in the ABCC11 gene is considered to be responsible for determination of earwax type. Cells with the A allele show a lower excretory activity for cGMP than those with the G allele. The A allele frequency shows a global North-South and East-West downward geographical gradient. Worldwide, it is highest in Chinese and Koreans where a dry earwax-type is retained amongst the various ethnic populations in these regions. Increased levels of axillary odour are associated with wet-type earwax, which is considered to be a direct consequence of axillary apocrine gland function.

Another protein which has been shown to play an important role in apocrine gland secretion is apolipoprotein D by Spielman, A. I. (1995) Experientia, 50: 40-47. This protein has been shown to act as a carrier vehicle for the abundant odour molecule E-3-methyl-2-hexanoic acid (3M2H). Studies have demonstrated that in apocrine secretions 3M2H is carried to the skin surface bound by two proteins, apocrine secretion odour-binding proteins 1 and 2 (ASOB1 and ASOB2). The ASOB2 protein was subsequently identified as apolipoprotein D (apoD), a known member of the α₂μ-microglobulin superfamily of carrier proteins also known as lipocalins (Zeng et al., (1996), 93: 6626-6630). Immunoreactivity for apoD has been localised to the apocrine glands in axillary tissue sections (Spielman et al., (1998) 134, 813-818) indicating that at least one of the glycoprotein carriers for 3M2H is localized in the apocrine glands.

Gross cystic disease fluid is a pathologic secretion from breast composed of several glycoproteins, including GCDFP-15. GCDFP-15 has been localized in the apocrine metaplastic epithelium lining breast cysts and in apocrine glands of the axilla, vulva, eyelid, and ear canal (Mazoujian et al., 1983 Am. J. Pathol., 110: 105-112). GCDFP-15 is identical to the Gp17/secretory actin binding protein (SABP/extra-parotid glycoprotein (EP-GP) which has been identified in the seminal vesicles, the salivary glands, and the sweat glands (Autiero et al., (1991); Exp. Cell Res., 197: 268-271), this protein is also identical to the prolactin inducible protein (PIP) (Murphy et al., (1987); J. Biol. Chem., 262: 15236-15241). GCDFP-15 is therefore a specific tissue marker of apocrine epithelium.

Zinc-α-glycoprotein (ZAG) is a 43 kDa soluble glycoprotein first isolated from human plasma (Burgi et al., (1961); J. Biol Chem; 236, 1066-1074) and subsequently found in secretory epithelia cells of liver, breast, the gastrointestinal tract, and sweat glands (Tada et al., (1991) J. Histochem Cytochem, 39, 1221-1226). ZAG is over-expressed in certain malignant tumours such that it may serve as a cancer maker (Diez-Itza et al., (1993), Eur. J. Cancer A 29, 1256-1260; Hale et al., (2001), Cancer Res., 7, 846-853). The biological functions of ZAG are largely unknown, but it has been shown to act as a lipid mobilizing factor (Todorov et al., (1998), Cancer Res., 58, 2353-2358) and a novel adipokine, which may be involved in the local regulation of adipose tissue function (Bao, et al., (2005) FEBS Letters, 579, 41-47).

As ABCC11, ApoD, GCDFP-15 and Zinc-α-glycoprotein play important roles in the secretion and transport processes of apocrine glands, we investigated the expression of transcripts responsible for the translation of these proteins in the indefinitely proliferating apocrine cell line.

The primers used to clone the gene transcript fragments are displayed in Table 6.

TABLE 6
Primers used to clone gene fragments involved
in apocrine secretory processes.

| | | Expected |
|---|---|---|
| | | size |
| Primer sequence | Gene | (bp) |
| Forward 5′- | ABCC11 | 254 |
| CACCATCCCTCCACTGTCAGTCC-3′ | | |
| Reverse 5′- | | |
| CAACATTCCCCAACTGCTCTTCTG-3′ | | |
| Forward 5′- | Apolipoprotein | 583 |
| CGGTGCGGCAGAGGGACAAG-3′ | D | |
| Reverse 5′- | | |
| GGGGAAAGCGAAGCAGAAGTAAC-3′ | | |
| Forward 5′- | GCDFP-15 | 340 |
| CCAGCCCTGCCACCCTGCTCC-3′ | | |
| Reverse 5′- | | |
| GCAGATGCCTAATTCCCGAATAAC-3′ | | |
| Forward 5′- | Zinc-α- | 518 |
| GGTGGAAGGAATGGAGGATTGG-3′ | glycoprotein | |
| Reverse 5′- | | |
| GTGGCAGGAGTAGGGGGCTG-3′ | | |

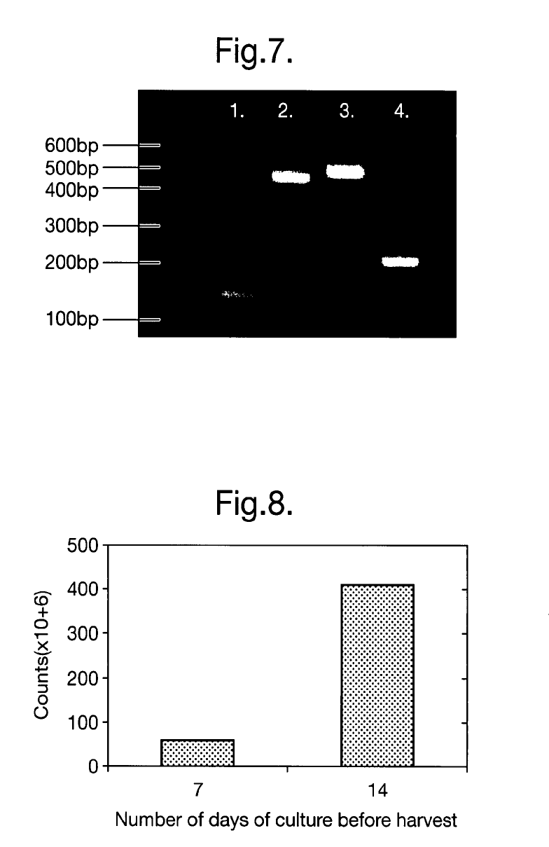

All the gene fragments that primer pairs were designed for, as outlined in table 6, could be detected. Appended FIG. 7 displays a 1.2% agarose gel of all the PCR products produced.

Appended FIG. 7. Agarose gel displaying the apocrine marker discloses: The following products are displayed in lanes 1 to 4 respectively.
1. ABCC11 transporter;
2. Apolipoprotein D;
3. Zinc-α-glycoprotein;
4. GCDFP-15

These PCR products were then cloned into the TOPO vector ready for sequencing. Sequencing was performed on each PCR product to confirm the identity of each cloned fragment. The results of the gene sequencing are given below.

ABCC11 fragment sequence
CACCATCCCTCCACTGTCAGTCCATGATGCCTCAGACAAAAATGTCCAAA
GGCTTCACCGTCTTTGGGAAGAAGAAGTCTCAAGGCGAGGGATTGAAAAA
GCTTCAGTGCTTCTGGTGATGCTGAGGTTCCAGAGAACAAGGTTGATTTT
CGATGCACTTCTGGGCATCTGCTTCTGCATTGCCAGTGTACTCGGGCCAA
TATTGATTATACCAAAGATCCTGGAATATTCAGAAGAGCAGTTGGGGAAT
GTTG
Apolipoprotein D fragment sequence
CGGTGCGGCAGAGGGACAAGCATTTCATCTTGGGAAGTGCCCCAATCCTC
CGGTGCAGGAGAATTTTGACGTGAATAAGTATCTCGGAAGATGGTACGAA
ATTGAGAAGATCCCAACAACCTTTGAGAATGGACGCTGCATCCAGGCCAA
CTACTCACTAATGGAAAACGGAAAGATCAAAGTGTTAAACCAGGAGTTGA
GAGCTGATGGAACTGTGAATCAAATCGAAGGTGAAGCCACCCCAGTTAAC
CTCACAGAGCCTGCCAAGCTGGAAGTTAAGTTTTCCTGGTTTATGCCATC
GGCACCGTACTGGATCCTGGCCACCGACTATGAGAACTATGCCCTCGTGT
ATTCCTGTACCTGCATCATCCAACTTTTTCACGTGGATTTTGCTTGGATC
TTGGCAAGAAACCCTAATCTCCCTCCAGAAACAGTGGACTCTCTAAAAAA
TATCCTGACTTCTAATAACATTGATGTCAAGAAAATGACGGTCACAGACC
AGGTGAACTGCCCCAAGCTCTCGTAACCAGGTTCTACAGGGAGGCTGCAC
CCACTCCATGTTACTTCTGCTTCGCTTTCCCC
GCDFP-15 fragment sequence
GCCCTTCCAGCCCTGCCACCCTGCTCCTGGTTCTCTGCCTGCAGTTGGGG
GCCAACAAAGCTCAGGACAACACTCGGAAGATCATAATAAAGAATTTTGA
CATTCCCAAGTCAGTACGTCCAAATGACGAAGTCACTGCAGTGCTTGCAG
TTCAAACAGAATTGAAAGAATGCATGGTGGTTAAAACTTACCTCATTAGC
AGCATCCCTCTACAAGGTGCATTTAACTATAAGTATACTGCCTGCCTATG
TGACGACAATCCAAAAACCTTCTACTGGGACTTTTACACCAACAGAACTG
TGCAAATTGCAGCCGTCGTTGATGTTATTCGGGAATTAGGCATCTGCAAG
GGC
Zinc-α-glycoprotein
GCCCTTGGTGGAAGGAATGGAGGATTGGAAGCAGGACAGCCAACTTCAGA
AGGCCAGGGNAGGACATCTTTATGGAGACCCTGAAAGACATTGTGGAGTA
TTACAACGACAGTAACGGGTCTCACGTATTGCAGGGAAGGTTTGGTTGTG
AGATCGAGAATAACAGAAGCAGCGGAGCATTCTGGAAATATTACTATGAT
GGAAAGGACTACATTGAATTCAACAAAGAAATCCCAGCCTGGGTCCCCTT
CGACCCAGCAGCCCAGATAACCAAGCAGAAGTGGGAGGCAGAACCAGTCT
ATGTGCAGCGGGCCAAGGCTTACCTGGAGGAGGAGTGCCCTGCGACTCTG
CGGAAATACCTGAAATACAGCAAAAATATCCTGGACCGGCAAGATCCTCC
CTCTGTGGTGGTCACCAGCCACCAGGCCCCAGGAGAAAAGAAGAAACTGA
AGTGCCTGGCCTACGACTTCTACCCAGGGAAAATTGGTGTGCACTGGACT
CGGGCCGGCGAGGTGCAGGAGCCTGAGTTACGGGGAGATGTTCTTCACAA
TGGAAATGGCACTTACCAGTCCTGGGTGGTGGTGGCAGTGCCCCCGCAGG
ACACAGCCCCCTACTCCTGCCACAAGGGC

The indefinitely proliferating apocrine cell line expresses the ABCC11 gene, which is associated with cellular secretory processes. The sequencing data confirmed the identity of the ABCC11 transporter gene. The genotype of the gene in this cell line is G (538G→A highlighted and underlined) and is associated with wet-earwax type and increased levels of axillary odour. The apolipoprotein D carrier gene is also expressed, indicating that the necessary machinery for the transport of odorants is also being manufactured in the cell line. The data provides further evidence of the apocrine phenotype of this cell line and its usefulness in providing a robust tool for the investigation of apocrine gland biology.

### Cell Chemical Composition—Materials and Methods

Indefinitely proliferating apocrine cells (passage 44) were harvested at 7 and 14 days, respectively. The cells were centrifuged at 1000 rpm for 5 mins washed once in PBS re-centrifuged and stored at −80° C. ready for analysis.

### 3M2H Glutamine Conjugate Analysis

3-Methyl-2-hexanoic acid glutamine conjugate was extracted from the frozen cell pellets using 10 μl (N-methyl-N-tert-butyldimethylsilyl) trifluoroacetamide (MTBSTFA), 1% tert-butyldimethylsilane (TBDMS) and 10 μl pyridine and 1 μl triethylamine. Derivatisation was carried out in the reagent on the samples. The mixture was maintained at 70° C. for 1 hour. The 3M2H glutamine derivatives were analysed by gas chromatography using an Agilent Technologies 6890/5973 gas chromatography/mass selective data system and an Agilent HP5-MS column (30 m×0.25 mm id×0.25 μm film) with selected ion monitoring, id indicating internal diameter. The temperature programme used was 70° C. to 270° C. at 10° C./min. Peak areas were calculated automatically using the in-built software. Structures were identified by mass spectrometry and retention times to known standards.

### Cholesterol and Squalene Analysis

Cholesterol and squalene were extracted from the frozen cell pellets using 20 μl of chloroform. This extract was then injected directly into the GC (details as above) and analysed under full scan using an Agilent HP5-MS column (30 m×0.25 mm id×0.25 μm film). Oven programme 70° C. to 270° C. at 10° C./min. Structures were identified by mass spectrometry and retention times to known standards.

### Short Chain Fatty Acids

Short chain fatty acids were extracted from the frozen cell pellets using 20 μl of chloroform. This extract was then injected directly into the GC (details as above) and analysed with selected ion monitoring using an HP-Innowax column (30 m×0.25 mm id×0.25 μm film). Oven programme 70° C. to 240° C. at 5° C./min. Structures were identified by mass spectrometry and retention times to known standards.

The relative levels of the 3M2H glutamine conjugate are shown in appended FIG. 8.

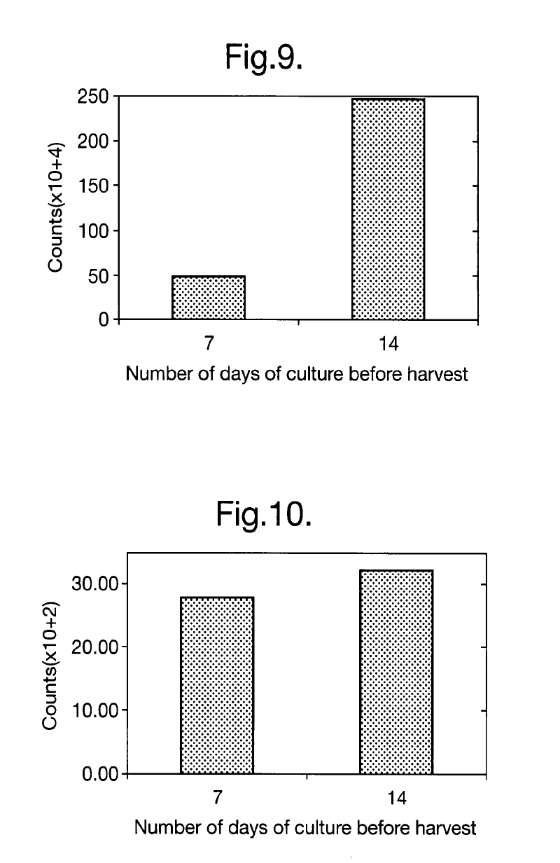

The relative levels of cholesterol in apocrine cells harvested after 7 and 14 days in culture are shown in appended FIG. 9.

The relative levels of squalene in apocrine cells harvested after 7 and 14 days in culture are shown in appended FIG. 10.

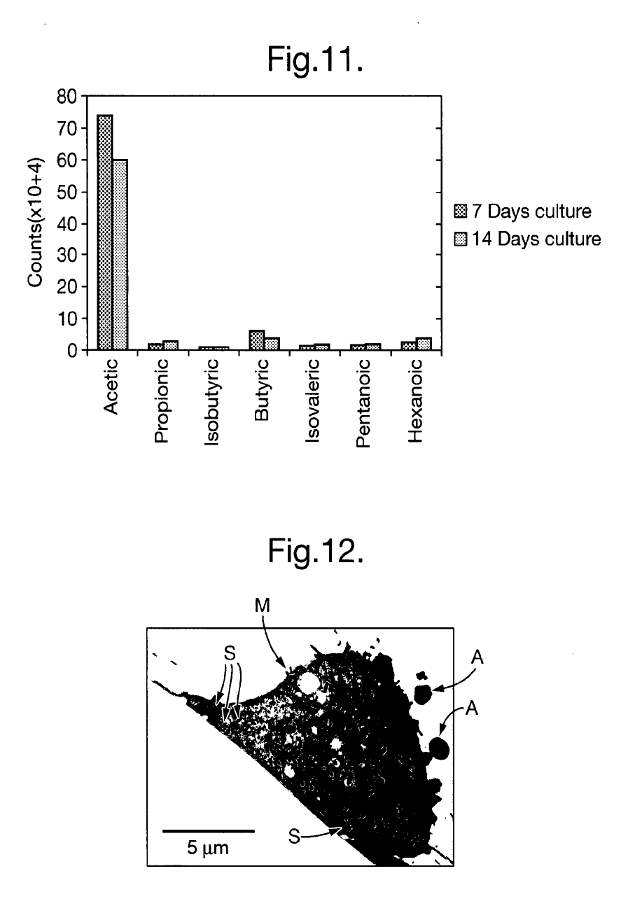

The relative levels of short chain fatty acids are shown in appended FIG. 11.

C16/18 fatty acids were also detected in the 14 day sample. The data summarised herein showing the presence of various volatile fatty acids demonstrates further that the indefinitely proliferating cell line mimicked successfully the functions of an apocrine gland.

### Inhibitor Studies

The production of malodour precursor compounds and the proliferation of cell growth would be expected to be modulated by the action of compounds known to interfere with androgen metabolizing enzymes. The proliferation of cells and the production of malodour precursor compounds would be compromised by the addition to the culture medium of antagonists known to interfere with androgen metabolising enzymes or with the androgen receptor directly. Such compounds would be expected to reduce the synthesis and secretion of odour precursor molecules both in culture and in apocrine glands residing in axillary skin. Such compounds include, but are not limited to:

17α-substituted benzylestradiols
Tricyclic coumarin sulfamates

### Estrone-3-O-sulfamate

Tricyclic oxepin sulfamate
2-methoxyestrone-3-O-sulfamate
Substituted chromenone sulfamates
17α-benzyl (or 4′-tert-butylbenzyl)estra-1,3,5(10)-trienes
17β(N-alkylcarbamoyl)-estra-1,3,5(10)-trien-3-O-sulfamates

### Trilostane

### Cyanoketone

Cyproterone acetate

### Norgestrel

### Norethindrone

### Thiazolidinediones

16-(bromoalkyl)-estradiols

### Flavonoids

### Isoflavonoids

### Lignans

Trifluoromethylacetylenic secoestradiol

6β-(thiaheptanamide) derivatives of estradiol

Estrone containing a spiro-gamma-lactone at position 17
7α-thioalkyl and 7α-thioaryl
Derivatives of spironolactone
N-butyl-N-methyl-11-(3′-hydroxy-21′,17′-carbolactone-19′-nor-17α-pregna-1′,3′,5′(10′)-trien-7′α-yl)-undecanamide
1,4-androstadiene-1,6,17-trione
Androsterone 33-substituted derivatives
4-azasteroids (MK386)
6-azasteroidal 17β-carboxamide triaryls
8-chloro-4-methyl-1,2,3,4,4a,5,6,10b-octaahydro-benzo[f]quinolin-3(2H)-one (LY 191704)

### 6-[4-(N,N-diisopropylcarbamoyl)phenyl]-N-methyl-quinolin-2-one 5)

Benzo[c]quinolizin-3-ones
Epicatechin-3-gallate
Epigallocatechin-3-gallate

### Suramin

### Zinc

Azelaic acid

### 6-[4-(N,N-diisopropylcarbamoyl)phenyl]-1H-quinolin-2-one 4

4-[3-[5-benzyl-8-(2-methyl)propyl-10,11-dihydrodibenz[b,f]azepine-2-carboxamido]phenoxy]butyric acid

### Turosteride

### MK-434

### Dihydrofinasteride

Chlormadinone acetate

### TZP-4238

### Epristeride (SK&F 105657, ONO-9302)

17α-estradiol
17-(5′-isoxazolyl)androsta-4,16-dien-3-one
N-(1,1,1,3,3,3-hexafluorophenyl-propyl)-3-oxo-4-aza-5α-androst-1-ene-17β-carboxamide (PNU 157706)

### Dutasteride

Oxendolone (TSAA-291: 16β-ethyl-17β-hydroxy-4-estren-3-one)

19-nor-10-azasteroids
Progesterone-based steroids bearing an oxime group
connected to the steroidal D-ring

### Benzoquinolinone

Serenoa repens extract permixon
Artocarpus incisus

### Alizarin

### Curcumin

Phenazine derivatives
Myristoleic acid
γ-linolenic acid
4-[3-[3-[bis(4-isobutylphenyl)-methylamino]benzoyl]-1H-indol-1-yl]butyric acid (FK 143)

### Flutamide

### Spironolactone

A number of compounds have been shown to reduce the proliferation of cells in the skin, including those cells originating from cutaneous appendages such as the sebaceous glands. Such compounds when contacted with the apocrine cells would be expected to reduce the synthesis and secretion of odour precursor molecules both in culture and in apocrine glands residing in axillary skin. Such compounds include, but are not limited to:

13-cis-retinoic acid
Retinoic acid

### Ultrastructural Analysis of Human Apocrine Cell Line ASG5 by Transmission Electron Microscopy (TEM)

### Sample Preparation

ASG5 cells passage number 47 were cultured in MEGM in a 95% air/5% CO₂ humidified incubator at 37° C., as described previously. Cells were harvested after 3 and 14 days incubation, respectively. Prior to fixation cells were washed twice in phosphate buffered saline pH 7.5.

### Transmission Electron Microscopy

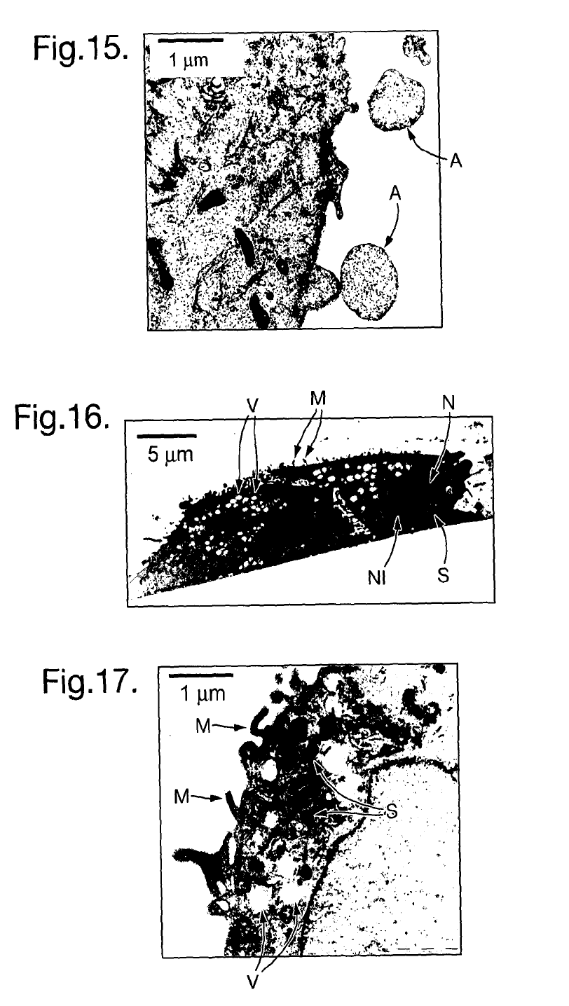

The washed cell cultures were fixed in 3% glutaraldehyde in 0.1 M cacodylate buffer pH 7.4 for 1 hour at room temperature. The specimens were washed in 0.1 M cacodylate buffer pH 7.4 for three periods of 5 minutes. After primary fixation specimens were fixed in 1% osmium tetroxide in 0.1 M cacodylate buffer pH 7.4 for 1 hour at room temperature. The specimens were washed again in 0.1 M cacodylate buffer pH 7.4 for three periods of 5 minutes. Specimens were then dehydrated through a grade series of ethanol. The cells were embedded in Luft's Epon resin and were polymerised at 60° C. for 48 hours. The blocks were sectioned on a Reichert “Ultracut S” Ultramicrotome set to give sections ca. 120 nm thick. The sections were picked up onto 200 mesh hexagonal thin bar copper grids. The sections were stained in uranyl acetate followed by lead citrate on a Leica EM stain. Stained sections were examined in a Philips CM120 transmission electron microscope operated at 120 kV. Images were recorded digitally, as .tif files and are shown herein as FIGS. 12 to 15 in respect of 3 day old culture cells and FIGS. 16 and 17 in respect of 14 day old culture cells.

### Results

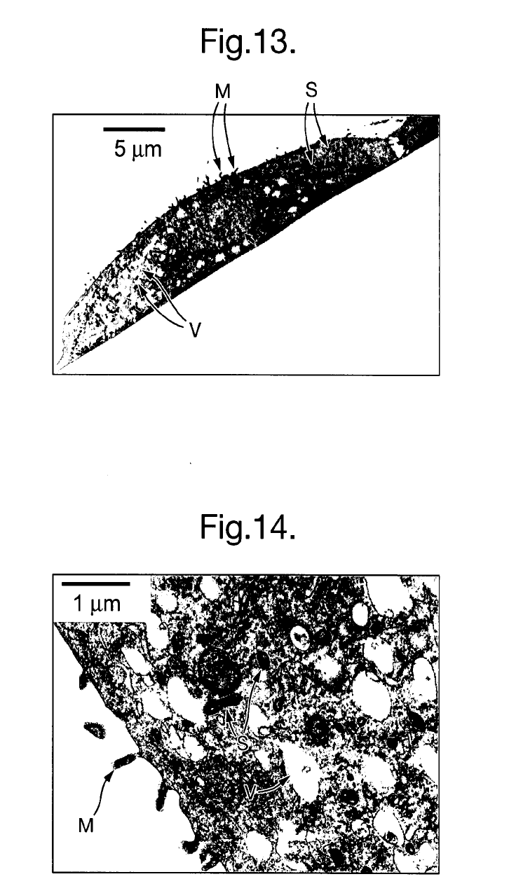

Cells shown in FIGS. 12, 13 and 16 exhibited a rounded or “cobblestone-like” appearance which is typical of epithelial cells in culture. The secretory nature of the cells was demonstrated by the presence of numerous microvilli identified as “M” (FIGS. 12, 13, 14 16 and 17) and apical blebs, designated as “A” (FIGS. 12, 15 and 16) at the luminal membrane, combined with the presence of numerous secretory granules, designated as “S”, throughout the cell cytoplasm (FIGS. 12 to 17). The pinching off of small portions of apical cytoplasm i.e. the microvilli “M” and apical blebs “A” constitute the apocrine secretion process (Montagna et al. (1953) Histology and cytochemistry of human skin. V. Axillary apocrine sweat glands. Am. J. Anat., 92: 451-470). All cells contained numerous secretory granules designated as “S” (FIGS. 12 to 17), although it was not possible to distinguish between Type I and Type II granules in accordance with (Bell (1974) The ultrastructure of human axillary apocrine glands after epinephrine injection. J. Invest. Dermatol., 63: 147-159). The granules contained highly electron-opaque particles, presumably containing small lipid droplets. Numerous “empty” vesicles, designated as “V”, were observed in most cells (FIGS. 12, 13, 14 16 and 17). This is probably as a consequence of the fixation process where the contents of the vesicles have been lost. Such vesicles are available to participate in the process of secretion. The cells in FIGS. 13 and 16 were rather flattened in appearance and clearly showed the nucleolus, designated “Nl”.

In all cells from both 3 and 14 day old ASG5 cultures apical blebs, “A”, luminal microvilli, “M” and numerous secretory granules, “S” were observed, confirming that these cells display typical active apocrine secretory morphology. These findings further substantiate the apocrine nature of the ASG5 cell line and provide additional evidence of the usefulness of this cell line as a functionally representative cell model of human axillary apocrine sweat glands.

1. An apocrine cell line exhibiting long term proliferation.

2. A cell line according to claim 1 derived from human apocrine glands

3. A cell line according to claim 1 that exhibits characteristic features of apocrine glands.

4. A cell line according to any preceding claim which is capable of proliferating indefinitely.

5. A cell line having the deposit number ECACC 07021301.

6. A method of obtaining a cultured cell line exhibiting long term proliferation comprising the steps of isolating an apocrine cell from primary tissue, culturing the isolated cell in a first culture medium, removing unattached cells from the first culture medium and transferring said unattached cells to a second culture medium comprising an effective concentration of a phorbol ester, and thereby establishing an apocrine cell line exhibiting long-term proliferation capability.

7. Use of an apocrine cell line according to any of claims 1 to 5 or produced according to claim 6 for diagnostic, therapeutic or cosmetic preparations.

8. Use of an apocrine cell line according to any of claims 1 to 5 or produced according to claim 6 for evaluating in vitro whether a material is a deodorant when topically applied to skin.

9. Use of an apocrine cell line according to any of claims 1 to 5 or produced according to claim 6 for the examination of the physiology or the pathophysiology of human apocrine gland.

10. Use of an apocrine cell line according to any of claims 1 to 5 or produced according to claim 6 for the testing of compounds or agents against diseases.

11. Use of an apocrine cell line according to any of claims 1 to 5 or produced according to claim 6 for the preparation of products derived from said cells.


SEQUENCE LISTING (39 sequences)

SEQ ID NO: 1 - DNA, 23 bp, Homo sapiens
cttcacccccaacttcaaccccg

SEQ ID NO: 2 - DNA, 23 bp, Homo sapiens
ccacatgcgtctgtctggtcccc

SEQ ID NO: 3 - DNA, 25 bp, Homo sapiens
gccagtcttcatctacaccagtagc

SEQ ID NO: 4 - DNA, 26 bp, Homo sapiens
ctctgtcatccttaaatcactgagtc

SEQ ID NO: 5 - DNA, 25 bp, Homo sapiens
cctcatccattgtaacatcacctcc

SEQ ID NO: 6 - DNA, 22 bp, Homo sapiens
ctcaccgcctggctacctgacc

SEQ ID NO: 7 - DNA, 24 bp, Homo sapiens
gacaagtgacagggaatggattcc

SEQ ID NO: 8 - DNA, 23 bp, Homo sapiens
cagggcttctggtagacatcagg

SEQ ID NO: 9 - DNA, 24 bp, Homo sapiens
gaagtgtgagtgcgcgaagatgcg

SEQ ID NO: 10 - DNA, 26 bp, Homo sapiens
gtgctggaattataggcatgagccac

SEQ ID NO: 11 - DNA, 21 bp, Homo sapiens
ccagccctggcgatggcaacg

SEQ ID NO: 12 - DNA, 26 bp, Homo sapiens
gaaattctgacctgttacacagtagg

SEQ ID NO: 13 - DNA, 24 bp, Homo sapiens
gcctgtcgtggacttggtcatgcg

SEQ ID NO: 14 - DNA, 24 bp, Homo sapiens
gagtaggtactgaccagccttctc

SEQ ID NO: 15 - DNA, 25 bp, Homo sapiens
cttggagagctgttggatggaggtg

SEQ ID NO: 16 - DNA, 22 bp, Homo sapiens
cagggccaggcgtcactgagac

SEQ ID NO: 17 - DNA, 23 bp, Homo sapiens
ctggatggggctcatggtgtttg

SEQ ID NO: 18 - DNA, 25 bp, Homo sapiens
gtttccaatgcttcactgggtgtgg

SEQ ID NO: 19 - DNA, 392 bp, Homo sapiens
tcccttcttcacccccaacttcaaccccgtgggttccaacggatgctttgccacacacgtgtgcttctgtttcgggagttatgtcacccatcacgacccacctttactctttgatatttccaaagatcccagagagagaaacccactaactccagcatccgagccccggttttatgaaatcctcaaagtcatgcaggaagctgcggacagacacacccagaccctgccagaggtgcccgatcagttttcatggaacaactttctttggaagccctggcttcagctgtgctgtccttccaccggcctgtcttgccagtgtgatagagaaaaacaggataagagactgagccgctagcagcgcctggggaccagacagacgcatgtggaagggc

SEQ ID NO: 20 - DNA, 813 bp, Homo sapiens
gcccttctctgtcatccttaaatcactgagtcttggacttcacggtctccttgtgccagtccacaagggaaacccactccacggttctaacagactcaggtacattcttcttggcttcctcccagctgtaaagtggcttatatgccagatcttgctgagccttcttgtaagagaaggtgaacacgctatttgacaatgtcactctgtggtggttgaagggtgtcgataggtgtaaattggcctcagcaggaagctcactatttccagcaggaagccaatccagtacatcagagataaaggaaggctccatctagaatcaaggcagaggccgaactctttgctcaggatgtaattaaggttatcatagctttggtgaggcatgtcatctgagatgtagtagaactgtcctcggacacttggggccttcttgggctcccgcagggcctcaaggccagaatgtgggcccaggccatgtttccaatatagactgggttgactctggagaacttgctgaaacttgacaggatcccatggttgtttagggcctcatttataccggcagaaaggattgggcttccttccccatagataaacattggtcttaaggcacaagtgtacaaggtgccaccgtttttcagagtccacccattagccgccagcacagccttctcagcaagctttttgctgtgtgggtatggagcgtaccatgtgttttccagaggctcttcttcgtgactgttctggatgatttccttgtaggaattgggcccggctacctctgggctactggtgtagatgaagactggcaagggc

SEQ ID NO: 21 - DNA, 502 bp, Homo sapiens
  misc_feature (14)..(14): n is a, c, g, or t
gcccttctcacccnctggctacctgaccttggtgttgagcttcaggtatgccacatagtgtgtcaggagcagcctttggaaggcaccgctgtagaaggcccaggccgggatcaggctcagaaagcccgccaagatttcatgggcaaggcagccgcaggtttcacctccaattgtgacataattcaatgactctttgacaaactcatcagcagtcttggttatcacatttgtatttagatactttgtcattgcagtcgggacagcatatggggtcagcacctggatgatgacttcttttgctttatattcctcttgcagggccttggaaaatgcgcacacaaacgccttggaagctgagtacatggagtagagaggccaaggaaacagggctatcccagaagaaatgttcaggatgagacctttctgccttgattccatatgtttcaaaattagctgtgtcatcttgactacggaggtgatgttacaatggatgaggaagggc

SEQ ID NO: 22 - DNA, 1024 bp, Homo sapiens
gacaagtgacagggaatggattccaaacaccagtgtgtaaagctaaaggatggccacttcatgcctgtattgggatttggcacctatgcacctccagaggttccaagaagtaaagctttggaggtcacaaaattagcaatagaagctgggttccgccatatagattctgctcatttatacaataatgaggagcaggttggactggccatccgaagcaagattgcagatggcagtgtgaagggagaagacatattctacacttcaaagctttggtccacttttcatcgaccagagttggtccgaccagccttggaaaactcactgaaaaaagctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcaggattggccaagtccattggggtgtcaaacttcaaccgcaggcagctggagatgatcctcaacaagccaggactcaagtacaagcctgtctgcaaccaggtagaatgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatattaacatggagagctttgcctgatgtctaccagaagccctg

SEQ ID NO: 23 - DNA, 1060 bp, Homo sapiens
gcccttgaagtgttagtgcgcgaagatgcgaaaggtggttttgatcaccggggctagcagtggcattggcctggccctctgcaagcggctgctggcggaagatgatgagcttcatctgtgtttggcgtgcaggaacatgagcaaggcagaagctgtctgtgctgctctgctggcctctcaccccactgctgaggtcaccattgtccaggtggatgtcagcaacctgcagtcggtcttccgggcctccaaggaacttaagcaaaggtttcagagattagactgtatatatctaaatgctgggatcatgcctaatccacaactaaatatcaaagcacttttctttggcctcttttcaagaaaagtgattcatatgttctccacagctgaaggcctgctgacccagggtgataagatcactgctgatggacttcaggaggtgtttgagaccaatgtctttggccattttatcctgattcgggaactggagcctctcctctgtcacagtgacaatccatctcagctcatctggacatcatctcgcagtgcaaggaaatctaatttcagcctcgaggacttccagcacagcaaaggcaaggaaccctacagctcttccaaatatgccactgaccttttgagtgtggctttgaacaggaacttcaaccagcagggtctctattccaatgtggcctgtccaggtacagcattgaccaatttgacatatggaattctgcctccgtttatatggacgctgttgatgccggcaatattgctacttcgcttttttgcaaatgcattcactttgacaccatataatggaacagaagctctggtatggcttttccaccaaaagcctgaatctctcaatcctctgatcaaatatctgagtgccaccactggctttggaagaaattacattatgacccagaagatggacctagatgaagacactgctgaaaaattttatcaaaagttactggaactggaaaagcacattagggtcactattcaaaaaacagataatcaggccaggctcagtggctcatgcctataattccagcac

SEQ ID NO: 24 - DNA, 918 bp, Homo sapiens
  misc_feature (911)..(911): n is a, c, g, or t
gcccttccagccctggcgatggcaacggcgacgggggtggcggaggagcgcctgctggccgcgctcgcctacctgcagtgcgccgtgggctgcgcggtcttcgcgcgcaatcgtcagacgaactcagtgtacggccgccccgcgtctccgcagcgcgcccaactgcatcctcctggccatgttcctcgtccactacgggcatcggtgcttaatttacccatttctgatgcgaggaggaaagcctatgccactgttggcgtgtacaatggcgattatgttctgtacctgtaacggctatttgcaaagcagatacttgagccattgtgcagtgtatgctgatgactgggtaacagatccccgttttctaataggttttggcttgtggttaacgggcatgttgataaacatccattcagatcatatcctaaggaatctcagaaaaccaggagatactggatacaaaataccaaggggaggcttatttgaatacgtaactgcagccaactattttggagaaatcatggagtggtgtggctatgccctggccagctggtctgtccaaggcgcggcttttgctttcttcacgttttgttttttatctggtagagcaaaagagcatcatgagtggtacctccggaaatttgaagagtatccaaagttcagaaaaattataattccatttttgttttaagtgcgtttttcatgaaattatcttcaacttgaagctttccaatggcgcttctctatggactttgtaaataagttatatctttgtaattttcctgctactttatcattttcaagatgtcctctaggaattttttttctagtaattttgcaatctacctaataagtacctaaatacgctgaaatggaggttgaatatcctactgtgtaacaggtcagaattncangggc

SEQ ID NO: 25 - DNA, 447 bp, Homo sapiens
  misc_feature (13)..(13): n is a, c, g, or t
gcccttgagtagntactgaccagccttctctagtgttccagacacctgtctgagtttcttggggtaaagatcatttccagcatgtttttagtctcatctgggtgcaaggacaagtcgtgtatcttctgtatgctctcaacacactgtccttgcaatgtcttcacgtggaatcgtctcagaagtgtaacgaggatggctttcatcatcaccatggcgatgtactttcctgcacagccacggggcccaaagccaaatggctgaaagtacctataaggaacattctttgcaaaattttcaagagtaaattcattgggtttggggaaaaactcgagtctgtgcatccttccaatattcaggataatgtttgtcccctttttcactgggtagccatcgattacatcatcttctaaggctttgcgcatgaccaagtccacgacaggcaagggc

SEQ ID NO: 26 - DNA, 564 bp, Homo sapiens
  misc_feature (13)..(13): n is a, c, g, or t
gcccttgtttccntgcttcactgggtgtggaaatagatgggcttgactttcccagaaaggatcttgggcacttgcacagagatgatctctgccatcatttccggaaagtccacgctcaccatgtgtgacttgattagcaggtcaaaagtgaactgatgcagctctctcgcaataggctgcacggagtccaggagcttggtgagctggtagaagcgtcttgagcaggatgtgggattttttcttttgcatgcaatgatacgatcgagttccttgatgtagttcattcgaagttcatcaaagaatttttgatttttcagcccatccactggaataatgctgaagagtagcagtgctttcatgcacaggaattcctggggggtgatttggagccatccaaactcttgagagaggtgcctcattcggacacactggctgtacatccgggacttgtgcatgcggtactcattgaaaaccagatcaggggcgaagtagagcatcctggagttgacattggtgaaggatcgccagcccatggcaaacaccatgagccccatccagaagggc

SEQ ID NO: 27 - DNA, 727 bp, Homo sapiens
  misc_feature (12)..(12): n is a, c, g, or t
gcccttcagggncnnggcgtcactgagactgtgggttctgggagccctctttgcttttactgtcctctgccgggctgcactcggaccccgtgatggaggacttgcacccgcgaagcacgtgggcattcagcatctccagcagcaggtcatacactgggaccacatttttgcacttcatgttgagcagatgttccatgcccttgttactcgcatgcctgacgtgggacaggagcatcaggaggttagccaggcgcatggattgctgctgggaggagatgccgctcttggcaatcacccaaaccaaagcatcggtcacggcgttcagcaagtgagccagcttccggctgctgtcagcatcctgggtcgctgtgaccagagggtacatactggaattgagcaggatcatggccttgacacagagatattctttgtgttggagttttaactctcgaaaccttgaagtagttgccaggagcatgtcaaagatttccagaattccttctacgcatttcccctcatccgtctgtaatcccaacaatttgggaggctgaggctgggggatcacttgaagtcaggacctcgagaccagcttggccaacatggtaaaaccccgtctccactgtccagaacaagatctggagcaaagatgagcctgccggggtggtcaattgagcgccacatcagccccatcattaacacctccatccaacagctctccaagaagggc

SEQ ID NO: 28 - DNA, 23 bp, Homo sapiens
caccatccctccactgtcagtcc

SEQ ID NO: 29 - DNA, 24 bp, Homo sapiens
caacattccccaactgctcttctg

SEQ ID NO: 30 - DNA, 20 bp, Homo sapiens
cggtgcggcagagggacaag

SEQ ID NO: 31 - DNA, 23 bp, Homo sapiens
ggggaaagcgaagcagaagtaac

SEQ ID NO: 32 - DNA, 21 bp, Homo sapiens
ccagccctgccaccctgctcc

SEQ ID NO: 33 - DNA, 24 bp, Homo sapiens
gcagatgcctaattcccgaataac

SEQ ID NO: 34 - DNA, 22 bp, Homo sapiens
ggtggaaggaatggaggattgg

SEQ ID NO: 35 - DNA, 20 bp, Homo sapiens
gtggcaggagtagggggctg

SEQ ID NO: 36 - DNA, 254 bp, Homo sapiens
caccatccctccactgtcagtccatgatgcctcagacaaaaatgtccaaaggcttcaccgtctttgggaagaagaagtctcaaggcgagggattgaaaaagcttcagtgcttctggtgatgctgaggttccagagaacaaggttgattttcgatgcacttctgggcatctgcttctgcattgccagtgtactcgggccaatattgattataccaaagatcctggaatattcagaagagcagttggggaatgttg

SEQ ID NO: 37 - DNA, 582 bp, Homo sapiens
cggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccaggccaactactcactaatggaaaacggaaagatcaaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtacctgcatcatccaactttttcacgtggattttgcttggatcttggcaagaaaccctaatctccctccagaaacagtggactctctaaaaaatatcctgacttctaataacattgatgtcaagaaaatgacggtcacagaccaggtgaactgccccaagctctcgtaaccaggttctacagggaggctgcacccactccatgttacttctgcttcgctttcccc

SEQ ID NO: 38 - DNA, 353 bp, Homo sapiens
gcccttccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgcaagggc

SEQ ID NO: 39 - DNA, 629 bp, Homo sapiens
  misc_feature (60)..(60): n is a, c, g, or t
gcccttggtggaaggaatggaggattggaagcaggacagccaacttcagaaggccagggnaggacatctttatggagaccctgaaagacattgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctatgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattggtgtgcactggactcgggccggcgaggtgcaggagcctgagttacggggagatgttcttcacaatggaaatggcacttaccagtcctgggtggtggtggcagtgcccccgcaggacacagccccctactcctgccacaagggc